(19) United States
(12) Patent Application Publication
(10) Pub. No.: US 2003/0073100 A1
(43) Pub. Date: Apr. 17, 2003

(54) Method of identifying renalgenerative agents using differential gene expression

(76) Inventors: John A. Peyman, New Haven, CT (US); Eero Lehtonen, Helsinki (FI); Oswald R. Crasta, Clinton, CT (US); Richard L. Cate, Cohasset, MA (US)

Correspondence Address: MINTZ, LEVIN, COHN, FERRIS,, GLOVSKY and POPEO, P.C., One Financial Center, Boston, MA, 02111, US

(21) Appl. No.: 10/113,312

(22) Filed: Apr. 1, 2002

Related U.S. Application Data
(60) Provisional application No. 60/280,258, filed on Mar. 30, 2001.

(51) Int. Cl. C12Q 1/68

(52) U.S. Cl. 435/6

(57) ABSTRACT

Disclosed are methods of identifying renalgenerative agensts using differential gene expression. Also disclosed are methods of treating renal disorders.

### RELATED U.S. APPLICATIONS

This application claims priority to U.S. Ser. No. 60/280,258 filed Mar. 30, 2001, which is incorporated herein by reference in its entirety.

### FIELD OF THE INVENTION

The invention relates generally to the identification of renalgenerative agents using differential gene expression.

### BACKGROUND OF THE INVENTION

Metanephrogenesis, definitive kidney formation, begins just before embryonic day 11 (E11). The ureteric bud (bud) grows about 200-300 μm from the nephric/Wolffian duct and invades the metanephrogenic mesenchyme (MM), a small, dense group of about 5000 cells within the intermediate mesoderm at the level of the hind limb. A few hours later, reciprocal induction interactions begin between bud epithelium and the MM. This occurs as a result of sequential activation of a series of genes from different families. These encode growth factors, receptors, oncoproteins, transcription factors, enzymes, signal transducers and extracellular matrix components.

Following induction (E10.75), the bud extends and bifurcates, acquiring a branching morphology by which it forms the collecting duct system. By E11.5, the MM first shows densely packed prenephrogenic cells and some looser mesenchymal prestromal components. Nephron formation starts at E12-13, when the essential features of the developing kidney are in place. The bud continues to arborise, while the dense mesenchyme surrounding its tips starts to form small condensations that will differentiate in to nephrons. At E13.5 begins the steady state of nephron morphogenesis, with prenephrogenic stem cells at the periphery. As maturing nephrons form, their loops of Henle descend into the developing medulla, and the collecting duct system expands its base, forming calyces. This reflects the start of kidney function at about E16. Kidney. Development continues after birth, with the loss of stem cells finally occurring about a month later.

### SUMMARY OF THE INVENTION

The invention is based in part on the discovery that certain nucleic acids are differentially expressed in metanephric mesenchyme undergoing mesenchymal-epithial transition (MET). These differentially expressed nucleic acids include nucleic acids sequences that, while previously described, have not heretofore been identified as mesenchymal-epithelial transition responsive.

In various aspects, the invention includes methods of identifying renalgenerative agents, methods of diagnosing renal disorders, and methods of treating renal disorders. For example, in one aspect, the invention provides a method of identifying a renalgenerative agent by providing a test cell population that includes one or more cells capable of expressing one or more nucleic acids sequences and contacting the test cell population with the test agent. Levels of expression of one or more sequences, termed MET sequences, are then compared to the levels of expression of the corresponding nucleic acids in a reference cell population. The reference cell population contains cells whose renalgenerative status is known, i.e., the reference cells are known to have been exposed to renalgenerative agent, or are known not to have been exposed to the renalgenrative agent.

In another aspect, the invention includes methods of treating a renal disorder or modulating kidney organogenesis in a subject by administering to the subject an agent that modulates the expression or activity of a renalgenerative genes (MET: 1-245).

The invention in a further aspect includes a method of selecting an individualized therapeutic agent appropriate for a particular subject. The method includes providing from the subject a test cell population comprising a cell capable of expressing one or more nucleic acids sequences responsive to renalgenerative agents, contacting the test cell population with the therapeutic agent, and comparing the expression of the nucleic acids sequences in the test cell population to the expression of the nucleic acids sequences in a reference cell population.

In a further aspect, the invention provides a method of diagnosing or determining susceptibility to a renal disorder, e.g. renal cancer, nephropathy, or nephritis. The method includes providing from the subject a cell population comprising a cell capable of expressing one or more renalgenerative genes, and comparing the expression of the nucleic acids sequences to the expression of the nucleic acids sequences in a reference cell population that includes cells from a subject not suffering from a renal disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention is based in part on the discovery of changes in expression patterns of multiple nucleic acid sequences in murine metanephric mesenchyme undergoing mesenchymal-epitheial transition (MET).

The differentially expressed nucleic acids were identified by inducing epithialization of murine metanephric mesenchyme explants. Metanephric mesenchymes were microdissected from day eleven mouse embroyos and the ureteric buds were removed. Spinal cords were collected as a source of factors for in vitro epithialization. Epithelialization of the metanephric mesenchyme was induced by a 48 trans-well co-culture with embryonic spinal cord tissue. (Lehtonen, et al., (2000) J Biol. Chem 275: 32888-32893.) Control samples included uninduced metanephric mesenchyme.

Genes whose transcript levels varied relative to the control samples were identified using GENECALLING™ differential expression analysis as described in U.S. Pat. No. 5,871,697 and in Shimkets et al., Nature Biotechnology 17:798-803 (1999). The contents of these patents and publications are incorporated herein by reference in their entirety.

Two hundred and forty five genes were found to be differentially expressed in epithelialized metanephric mesenchyme. These sequences are referred to herein as MET 1-261 A summary of the MET sequences analyzed is presented in Tables 1 and 2 One hundred and forty eight genes were upregulated as shown in Table 1. Ninety seven genes were downregulated as shown in Table 2.

For a given MET sequence, its expression can be measured using any of the associated nucleic acid sequence in the methods described herein. For previously described sequences (MET:1-245), database accession numbers are provided. This information allows for one of ordinary skill in the art to deduce information necessary for detecting and measuring expression of the MET nucleic acid sequences.

The mesenchymal-epithelial transition responsive nucleic acids discussed herein include the following:

TABLE 1
Mesenchymal-Epithelial Transition Upregulated Nucleic Acids

| | | | |
|---|---|---|---|
| GenBank | | MET | |
| AccNo | Encoded Protein | Assignment | Fold Induction |

Membrane Proteins

| | | | |
|---|---|---|---|
| af031573 | Flamingo 1 | 1 | 3.73 |
| ab017202 | Entactin-2 | 2 | 3.24 |
| u67399 | K-cadherin/cadherin-6 | 3 | 2.95 |
| ak014622 | Pdk1, polycystic kidney disease 1 | 4 | 2.22 |
| m58156 | Mhc (A.CA/J(H-2K-f) class I antigen | 5 | 2.20 |
| ai315935 | Tnf-R, tumor necrosis factor receptor 55 KD | 6 | 2.13 |
| af035814 | Bec1, voltage-gated K(+) channel | 7 | 2.10 |
| u95030 | Leukocyte cell adhesion molecule CD166 (ALCAM) | 8 | 2.09 |
| af029694 | Extracellular matrix protein (Ecm1) p85 | 9 | 2.06 |
| ai385828 | Ptk7, homolog of human transmembrane receptor | 10 | 2.02 |
| u55057 | Ptp-lambda, receptor protein tyrosine phosphatase-lamda | 11 | 2.00 |
| y00051 | Ncam, neural cell adhesion molecule | 12 | 1.97 |
| aa987131 | Homolog of rat calcium-independent alpha-latrotoxin receptor | 13 | 1.96 |
| aj243651 | Irt1, homolog of putative metal transporter | 14 | 1.84 |
| m33760 | Fgf-R1, fibroblast growth factor receptor 1 | 15 | 1.82 |
| u64199 | I1-12-R beta2, interleukin-12 receptor beta2 | 16 | 1.76 |
| u12983 | Cek5 receptor protein-tyrosine kinase | 17 | 1.74 |
| ak002599 | Integral membrane protein 2 B | 18 | 1.74 |
| x59560 | Tyrosine kinase receptor | 19 | 1.73 |
| bb389565 | Homolog of rat neurotrimin | 20 | 1.69 |
| be631582 | Cd27.homolog of human CD27, TNF receptor family | 21 | 1.67 |
| s57168 | Sek, Eph-related receptor protein tyrosine kinase | 22 | 1.63 |
| u76253 | E25b protein | 23 | 1.61 |
| ab031959 | Lst-1, Liver transporter | 24 | 1.59 |
| u54984 | Mt1-Mmp, membrane-type matrix metalloproteinase 1 | 25 | 1.56 |
| m25149 | Tum-P91A antigen | 26 | 1.55 |
| u06834 | Eph-related receptor protein tyrosine kinase | 27 | 1.53 |
| ak004593 | K+ channel tetramerisation domain-containing tumor necrosis factor alpha- | 28 | 1.53 |
| | induced protein 1 | | |
| sc-120308535 | GlyCAM1, endothelial ligand for L-selectin | 29 | 1.52 |
| af192310 | Neutral- and basic-amino-acid transporter heavy-chain | 30 | 1.51 |

Secreted Proteins

| | | | |
|---|---|---|---|
| j03484 | Laminin B2 chain | 32 | 11.57 |
| aw909815 | Homolog of human small Ca-dependent protease | 33 | 7.90 |
| u34606 | Collagen alpha 1(XVIII) | 34 | 5.07 |
| x70854 | Fibulin D form | 35 | 4.89 |
| l20276 | Biglycan (Bgn) | 36 | 4.14 |
| u18746 | Fibroblast growth factor | 37 | 3.95 |
| af082859 | Lungkine (Weche) | 38 | 3.67 |
| l16898 | Collagen alpha 1(XVIII) | 39 | 3.50 |
| m89797 | Wnt4 | 40 | 3.37 |
| af176645 | Collagen alpha 3 (V) | 41 | 3.09 |
| x58251 | Collagen alpha 2 (I) | 42 | 2.97 |
| u08020 | Collagen alpha 1 (I) | 43 | 2.59 |
| d50462 | Sdf5, homologs of rat frizzled (fz-1 and fz-2) | 44 | 2.52 |
| j04694 | Collagen alpha 1 (IV) | 45 | 2.50 |
| x04647 | Collagen alpha 2 (IV) | 46 | 2.31 |
| x70296 | PN-1, protease-nexin 1 | 47 | 2.29 |
| bf118182 | Collagen alpha 2 (V) | 48 | 2.22 |
| u48854 | Dag1, dystroglycan 1 | 49 | 2.21 |
| m84324 | Collagenase type IV | 50 | 2.21 |
| u69176 | Laminin alpha 4 | 51 | 2.09 |
| sc-120993069 | Protocadherin 2 | 52 | 2.01 |
| m91380 | Tsc-36, TGF-beta-inducible protein | 53 | 1.99 |
| x04017 | Sparc, cysteine-rich glycoprotein, osteonectin | 54 | 1.99 |
| u34277 | PAF acetylhydrolase | 55 | 1.91 |
| aa536781 | Agrin | 56 | 1.77 |
| l12215 | Collagen alpha 1 (IX) | 57 | 1.73 |
| l12447 | Igfbp-5, insulin-like growth factor binding protein 5 | 58 | 1.69 |
| l47480 | Bmp-4, bone morphogenetic protein 4 | 59 | 1.68 |
| y12582 | Calpain-like protease | 60 | 1.68 |
| af144628 | Slit2 | 61 | 1.67 |
| x68378 | Cathepsin D | 62 | 1.61 |
| x81582 | Igfbp-4, insulin-like growth factor binding protein 4 | 63 | 1.58 |
| x80992 | Vgr-1 | 64 | 1.57 |
| ab008548 | Type 1 procollagen C-proteinase enhancer | 65 | 1.56 |
| u71085.1 | Igf2, insulin-like growth factor 2 | 66 | 1.56 |
| af006465 | Ibap-1, B cell Ag-receptor Ig beta assoc. protein 1 | 67 | 1.50 |
| bf227184 | Kappa casein | 68 | 1.50 |
| u37459 | GDNF, glial derived neurotrophic factor (10 frag.) | 69 | 1.06 |
| d10213 | HGF, hepatocyte growth factor (19 cDNA fragments) | 70 | 1.06 |
| x51801 | Bmp-7, bone morphogenetic protein-7 (7 fragments) | 71 | 1.00 |

Receptor Signaling

| | | | |
|---|---|---|---|
| aj251859 | Pde7, phosphodiesterase 7 | 72 | 2.35 |
| af176903 | Sprouty1 | 73 | 2.06 |
| af026216 | Mkk7, mitogen-activated protein kinase kinase | 74 | 1.98 |
| x13460 | Annexin I | 75 | 1.93 |
| u36488 | Stem cell phosphatase (Esp) | 76 | 1.91 |
| af191839 | Tbk1, TANK binding kinase | 77 | 1.88 |
| ak010660 | LIM domain containing protein | 78 | 1.84 |
| aa289577 | Inpp5p II, inositol polyphosphate 5-phosphatase II | 79 | 1.81 |
| l22482 | Hic5, paxillin-like protein | 80 | 1.69 |
| x85983 | Carnitine acetyltransferase | 81 | 1.65 |
| af240630 | Iqgap1, IQ motif containing GTPase activating protein 1 | 82 | 1.65 |
| m74227 | Cyclophilin C | 83 | 1.61 |
| ai385900 | S6 kinase, ribosomal protein S6 kinase | 84 | 1.61 |
| u88327 | Socs-2, suppressor of cytokine signaling-2 | 85 | 1.57 |
| d11374 | Spa1, GTPase-activating protein | 86 | 1.53 |

Cytoskeleton

| | | | |
|---|---|---|---|
| m90365 | Catenin gamma, plakoglobin | 87 | 2.09 |
| af144095 | Myosin 15, unconventional myosin | 88 | 1.94 |
| s76831 | Tropomodulin | 89 | 1.72 |
| af104414 | Large tumor suppressor 1 (Lats1) | 90 | 1.70 |
| ak014169 | Ch-tog, colon/hepatic tumor over-expressed gene | 91 | 1.64 |
| an218430 | Dynein | 92 | 1.63 |
| aa870409 | Homolog of human endothelial actin-binding protein | 93 | 1.56 |

Transcription Factors and Nuclear Proteins

| | | | |
|---|---|---|---|
| x55781 | Pax2, embryonic transcription factor | 94 | 2.48 |
| x94127 | Homolog of human Sox2, embryonic transcription factor | 95 | 2.28 |
| l17069 | All-1, homolog of drosophila Ash 1 chromatin remodeling protein | 96 | 2.02 |
| ab006360 | Pur-1, Zn-finger protein | 97 | 2.00 |
| u02278 | Hox B3, homeobox B3 protein | 98 | 1.98 |
| aw990233 | Tgif, DNA-binding homeodomain protein | 99 | 1.88 |
| ak017894 | Transcription factor with bromodomain adjacent to zinc finger domain | 100 | 1.88 |
| d31967 | Jmj, jumonji protein | 101 | 1.87 |
| aa286377 | Homolog of human WBSCR9 chromatin remodeling protein | 102 | 1.86 |
| ai323617 | Mef2, myocyte enhancer factor-2 | 103 | 1.81 |
| l38620 | Sin3A transcriptional co-repressor | 104 | 1.78 |
| aw557731 | Pnuts, homolog of rat putative protein phosphatase 1 nuclear targeting | 105 | 1.75 |
| | subunit | | |
| d55720 | Importin alpha | 106 | 1.74 |
| af237703 | RNA polymerase I transcription termination factor 1 | 107 | 1.73 |
| x83974 | Ttf1, ribosomal gene Pol I transcription termination factor | 108 | 1.73 |
| af000938 | Rpa I, RNA polymerase I largest subunit | 109 | 1.70 |
| ab043550 | Narf, neural activity-related ring finger protein | 110 | 1.70 |
| aj007396 | Sal-3, spalt-like Zn-finger protein, homolog of human SAL-2 | 111 | 1.68 |
| af315352 | Nocturnin, leucine zipper protein | 112 | 1.66 |
| ak011832 | Zinc finger protein 131 homolog | 113 | 1.62 |
| af182040 | Cbfl, Suppressor of Hairless [Su(H)]/Lag-1/RBP-Jkappa | 114 | 1.54 |
| m55512 | WT-1, Willms' tumor gene-1, 4 cDNA fragments | 115 | 1.21 |

Nuclear receptors

| | | | |
|---|---|---|---|
| u07635 | Arp-1, apolipoprotein regulatory protein-1, orphan nuclear receptor in the | 116 | 1.55 |
| | COUP family | | |

Endoplasmic Reticulum, Golgi, Lysosome

| | | | |
|---|---|---|---|
| sc-121025646 | Homolog of human KDEL-receptor | 117 | 3.13 |
| u34259 | Mtp, Membrane-Lysosome-Golgi 4-TM transporter | 118 | 2.80 |
| j05287 | Lamp-2, lysosome-associated membrane protein | 119 | 2.14 |
| d13003 | Reticulocalbin | 120 | 1.94 |
| ab011451 | SulT, GlcNAc-sulfotransferase | 121 | 1.92 |
| af279263 | Fkbp65rs, ER-localized chaperone | 122 | 1.78 |
| u72141 | Etx2, multiple exostosis protein | 123 | 1.71 |
| af218084 | Rep1, rab escort protein 1, choroideremia protein chm | 124 | 1.70 |
| be456824 | Rer1, homolog of yeast Sec12p protein, ER protein | 125 | 1.62 |
| j05185 | Pdi, protein disulfide isomerase | 126 | 1.57 |
| aw762467 | GlcNac T, beta-1,3-N-acetylglucosaminyltransferase | 127 | 1.53 |
| aa472331 | Gst, glutathione S-transferase | 128 | 1.51 |

Metabolic Enzymes and Other Proteins

| | | | |
|---|---|---|---|
| ai648997 | Atpsk2, ATP sulfurylase/APS kinase 2 | 129 | 6.26 |
| x13135 | Fas, fatty acid synthase | 130 | 2.48 |
| af216873 | AcetylCoA Synthetase | 131 | 2.33 |
| x06917 | Ast, aspartate aminotransferase | 132 | 2.01 |
| d28529 | Ptp-bl, protein tyrosine phosphatase | 133 | 1.90 |
| af176524 | Fbl10, F-box protein | 134 | 1.88 |
| ab036882 | Midnolin, midbrain nucleolar protein | 135 | 1.76 |
| af064635 | Kik-I, putative steroid dehydrogenase | 136 | 1.72 |
| aa619781 | Alt, homolog of human alanine aminotransferase | 137 | 1.70 |
| af156987 | Cry2, cryptochrome 2 | 138 | 1.69 |
| au078836 | Fadsd5, delta-5 fatty acid desaturase | 139 | 1.67 |
| af172275 | Fus/Tls, oncogene/splicing factor | 140 | 1.66 |
| bf016476 | Homolog of human eIF-4B, translation initiation factor | 141 | 1.65 |
| aw540167 | SAM synthetase, S-adenosylmethionine synthetase | 142 | 1.65 |
| m26270 | Scd2, stearoyl-CoA desaturase | 143 | 1.62 |
| aa119427 | Homolog of human G6PI, glucosamine-6-phosphate isomerase | 144 | 1.61 |
| aa516578 | Hsp25, heat shock protein 25 | 145 | 1.57 |
| aa473691 | eIF-4-gamma, eukaryotic translation initiation factor | 146 | 1.55 |
| aa545101 | Slu7-like protein, splicing factor | 147 | 1.52 |
| af177346 | Plic-2, proteins linking IAP (integrin-associated protein (CD47)) with | 148 | 1.50 |
| | cytoskeleton | | |

TABLE 2
Mesenchymal-Epithelial Transition Downregulated Nucleic Acids

| | | | |
|---|---|---|---|
| GenBank | | MET | |
| AccNo | Encoded Protein | Assignment | Fold Suppression |

Membrane Proteins

| | | | |
|---|---|---|---|
| z12171 | Dlk, delta-like | 149 | −1.53 |
| bb097076 | Edg2, homolog of rat G10 protein | 150 | −1.58 |
| x84037 | E-selectin ligand-1 | 151 | −1.59 |
| af240002 | Ant1, adenine nucleotide translocase 1 | 152 | −1.61 |
| m29379 | Anion exchange protein | 153 | −1.63 |
| af335543 | H47, minor histocompatibility antigen precursor | 154 | −1.73 |
| af078748 | Slc22a3, organic cation transporter 3 | 155 | −1.83 |
| aa163461 | Flrt3, homolog of human leucine-rich repeat transmembrane protein | 156 | −1.86 |
| af096875 | Type 2 iodothyronine deiodinase | 157 | −1.86 |
| j04634 | Cell surface antigen 114/A10 | 158 | −1.94 |
| ab022913 | Glutamate receptor channel alpha4 | 159 | −1.94 |
| bb453301 | Homolog of rat Trk1, neurotrophin receptor | 160 | −2.27 |
| m23384 | Glucose transporter type 1 | 161 | −2.61 |
| af062476 | Retinoic acid-responsive protein (Stra6) | 162 | −3.50 |
| aa575705 | Homolog of rat neurotransmitter transporter RB21A | 163 | −3.54 |

Secreted Proteins

| | | | |
|---|---|---|---|
| aa537293 | Homolog of human fibromodulin | 164 | −1.53 |
| af013262 | Ldc, lumican | 165 | −1.53 |
| aw702074 | Lactotransferrin | 166 | −1.55 |
| ab046417 | Phosphatidylethanolamine binding protein | 167 | −1.61 |
| af105268 | Gpc6, glypican-6 | 168 | −1.61 |
| af251024 | Chaperonin 10 | 169 | −1.63 |
| ab037111 | Neutral ceramidase | 170 | −1.79 |
| aa798928 | Serum protease MSE55 | 171 | −1.85 |
| aa098478 | Sdf1, stromal derived factor 1 | 172 | −1.90 |
| ak008922 | Homolog of FGF 10, fibroblast growth factor 10 | 173 | −2.07 |
| s78114 | Surfactant protein B | 174 | −2.27 |
| m57625 | Protease-6 | 175 | −2.56 |
| l03799 | Ice, interleukin-1 converting enzyme | 176 | −2.60 |
| bf321311 | Erp, non-transmembrane tyrosine phosphatase | 177 | −3.36 |
| m32490 | Cyr61 | 178 | −3.54 |
| ai195609 | Dbp, vitamin D binding protein | 179 | −12.61 |

Receptor Signaling

| | | | |
|---|---|---|---|
| m96163 | Serum inducible kinase (SNK) | 180 | −1.51 |
| af145285 | G3bp-2a, RNA-binding protein | 181 | −1.54 |
| u72059 | Icln, chloride conductance regulatory protein | 182 | −1.55 |
| ai451770 | Homolog of human Rap1 GTPase activating protein | 183 | −1.59 |
| bb186331 | Nemo, NF-kappaB essential modulator, IKKgamma | 184 | −1.59 |
| af100694 | Pontin52 | 185 | −1.61 |
| aa684197 | Cl-6, homolog of rat growth response protein | 186 | −1.63 |
| x79082 | Ebk receptor tyrosine kinase | 187 | −1.64 |
| af143956 | Coronin-2 | 188 | −1.67 |
| af126543 | 100 kDa thyroid hormone receptor associated protein | 189 | −1.67 |
| x16857 | Hsp86, heat shock protein 86 | 190 | −1.69 |
| au051751 | Ca-binding protein A10, annexin II ligand, calpactin I | 191 | −1.73 |
| af187066 | p75NTR-associated cell death executor (Nade) | 192 | −1.85 |
| x99963 | Rho B | 193 | −1.97 |
| u28495 | Lfc oncogene | 194 | −2.37 |
| aw912085 | Pim-1 serine kinase | 195 | −2.59 |
| u11054 | Nuclear dual specificity kinase Sty | 196 | −2.82 |
| s64851 | Erp tyrosine phosphatase, map kinase phosphatase 1, dual specificity | 197 | −3.36 |
| | phosphatase | | |

Cytoskeleton

| | | | |
|---|---|---|---|
| aa537605 | Capping protein beta-subunit isoform 1 | 198 | −1.50 |
| aa537141 | Myosin light chain 3, non-muscle myosin | 199 | −1.51 |
| af233340 | Synbindin | 200 | −1.59 |
| ak008947 | Coronin | 201 | −1.67 |
| x72711 | Replication factor C, large subunit | 202 | −1.51 |
| d86725 | MCM2/BM28, minichromosome maintenance 2 | 203 | −1.56 |
| ai528428 | Variant histone H3.3 | 204 | −1.61 |
| af294327 | Ran binding protein 5 | 205 | −1.67 |
| l16846 | Btg1, B-cell translocation gene-1 protein | 206 | −1.68 |
| u48853 | p130Cas, Crk-associated substrate | 207 | −1.94 |
| s71186 | Xpbc/ercc-3 DNA repair gene | 208 | −3.54 |
| x57487 | Pax8 (4 cDNA fragments) | 209 | −1.05 |

Transcription Factors and Nuclear Proteins

| | | | |
|---|---|---|---|
| af079852 | IKLF, intestinal-enriched Krueppel-like factor, Krueppel-like factor V | 210 | −1.50 |
| m88489 | Vdjp, Nonamer binding prote+J167in, similar to Rep C | 211 | −1.51 |
| l38607 | BF-2 transcription factor | 212 | −1.59 |
| u32395 | Mad4, Max-interacting transcriptional repressor | 213 | −1.60 |
| l13791 | C/ATF, CCAAT/enhancer binding proteins related activating transcription | 214 | −1.61 |
| | factor | | |
| u87620 | Spi-B, Ets transcription factor | 215 | −1.61 |
| ab029448 | Dlxin-1, regulator of Dlx5 homeodomain protein | 216 | −1.62 |
| ak005204 | Homolog of human highly charged adrenal protein | 217 | −1.73 |
| m69293 | Id2, basic helix-loop-helix transcription factor | 218 | −1.83 |
| af097440 | Bex3, brain expressed X-linked protein 3 | 219 | −1.85 |
| af098967 | Ilf3, interleukin enhancer binding factor 3 | 220 | −2.42 |
| aw210091 | Tilz2, TSC22-related inducible leucine zipper 2 | 221 | −2.51 |

Nuclear Receptor

| | | | |
|---|---|---|---|
| aw910999 | Trap100, thyroid hormone receptor-associated protein | 222 | −1.67 |

Endoplasmic Reticulum, Golgi, Lysosome

| | | | |
|---|---|---|---|
| ak011276 | Homolog of human ER transmembrane protein | 223 | −1.57 |
| aa529571 | Bip, 78 kDa glucose-regulated protein | 224 | −1.65 |
| d84436 | PigB, glycosylphosphatidylinositol (GPI) anchor addition B | 225 | −3.37 |

Metabolic Enzymes and Other Proteins

| | | | |
|---|---|---|---|
| aa987153 | Sahh, S-adenosylhomocysteine hydrolase | 226 | −1.63 |
| ak006441 | Gpx-4, glutathione peroxidase 4 | 227 | −1.63 |
| af129888 | Sui1, translation initiation factor | 228 | −1.71 |
| j04633 | Hsp84, heat shock protein 84 | 229 | −1.73 |
| x99273 | Raldh, retinaldehyde-specific dehydrogenase | 230 | −1.75 |
| m22873 | eIF-4A, Initiation factor eIF-4A | 231 | −1.78 |
| aa450670 | Pgm, phosphoglycerate mutase | 232 | −1.78 |
| aa272819 | GalK, galactokinase | 233 | −1.84 |
| m32599 | Gapdh, glyceraldehyde-3-phosphate dehydrogenase | 234 | −1.86 |
| sc-121021927 | Pk-M, pyruvate kinase M | 235 | −1.99 |
| x97752 | Rdh, 11-cis retinol dehydrogenase | 236 | −2.27 |
| bb044864 | Star, steroidogenic acute regulatory protein | 237 | −2.27 |
| ai788340 | Homolog of rat trans-Golgi protein GMx33 | 238 | −2.52 |
| x61600 | Beta-enolase | 239 | −2.81 |
| x80852 | Pfk, phosphofructokinase | 240 | −2.92 |
| ab047323 | Cox17p, copper chaperone protein | 241 | −3.16 |
| ac002393.6 | Tpi, triose phosphate isomerase | 242 | −3.43 |
| au080370 | SelW, selenoprotein W | 243 | −3.46 |
| m23961 | Pgk1-ps1, phosphoglycerate kinase processed pseudogene | 244 | −4.06 |
| | Zeta-globin | 245 | −15.02 |

General Screeening and Diagnostic Methods Using MET Sequences

Several of the herein disclosed methods relate to comparing the levels of expression of one or more MET nucleic acids in a test and reference cell populations. The sequence information disclosed herein, coupled with nucleic acid detection methods known in the art, allow for detection and comparison of the various MET transcripts. In some embodiments, the MET nucleic acids and polypeptide correspond to nucleic acids or polypeptides which include the various sequences (referenced by SEQ ID NOs) disclosed for each MET nucleic acid sequence.

In its various aspects and embodiments, the invention includes providing a test cell population which includes at least one cell that is capable of expressing one or more of the sequences MET 1-245, or any combination of MET sequences thereof. By “capable of expressing” is meant that the gene is present in an intact form in the cell and can be expressed. Expression of one, some, or all of the MET sequences is then detected, if present, and, preferably, measured. Using sequence information provided by the database entries for the known sequences, or the sequence information for the newly described sequences, expression of the MET sequences can be detected (if expressed) and measured using techniques well known to one of ordinary skill in the art. For example, sequences within the sequence database entries corresponding to MET sequences, or within the sequences disclosed herein, can be used to construct probes for detecting MET RNA sequences in, e.g., northern blot hybridization analyses or methods which specifically, and, preferably, quantitatively amplify specific nucleic acid sequences. As another example, the sequences can be used to construct primers for specifically amplifying the MET sequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

For MET sequences whose polypeptide product is known, expression can be also measured at the protein level, i.e., by measuring the levels of polypeptides encoded by the gene products described herein. Such methods are well known in the art and include, e.g., immunoassays based on antibodies to proteins encoded by the genes.

Expression level of one or more of the MET sequences in the test cell population is then compared to expression levels of the sequences in one or more cells from a reference cell population. Expression of sequences in test and control populations of cells can be compared using any art-recognized method for comparing expression of nucleic acid sequences. For example, expression can be compared using GENECALLING™ methods as described in U.S. Pat. No. 5,871,697 and in Shinikets et al., Nat. Biotechnol. 17:798-803.

In various embodiments, the expression of 2, 3, 4, 5, 6, 7, 8, 9, 10, 15, 20, 50, 100, 150, 200, or all of the sequences represented by MET 1-245 are measured. If desired, expression of these sequences can be measured along with other sequences whose expression is known to be altered according to one of the herein described parameters or conditions.

The reference cell population includes cells one or more cells capable of expressing the measured MET sequences and for which the compared parameter is known, e.g., prenalgenerative agent exposure status. By “renalgenerative agent exposure status” is meant that it is know whether the reference cell has been exposed to a renalgenerative agent. A renalgenrative agent is an agent that induces the transistion of metanephric mesenchyme to epithelial cells. An example of a renalgenerative agent includes spinal cord tissue. Whether or not comparison of the gene expression profile in the test cell population to the reference cell population reveals the presence, or degree, of the measured parameter depends on the composition of the reference cell population. For example, if the reference cell population is composed of cells that have not been treated with a renalgenerative agent, a similar gene expression level in the test cell population and a reference cell population indicates the test agent is not a renalgenerative agent. Conversely, if the reference cell population is made up of cells that have been treated with a renalgenerative agent, a similar gene expression profile between the test cell population and the reference cell population indicates the test agent is a renalgenerative agent.

In various embodiments, a MET sequence in a test cell population is considered comparable in expression level to the expression level of the MET sequence in the reference cell population if its expression level varies within a factor of 2.0, 1.5, or 1.0 fold to the level of the MET transcript in the reference cell population. In various embodiments, a MET sequence in a test cell population can be considered altered in levels of expression if its expression level varies from the reference cell population by more than 1.0, 1.5, 2.0 or more fold from the expression level of the corresponding MET sequence in the reference cell population. In some embodiments, the variation in expression of a particular MET sequence corresponds to the change in expression level observed for the MET sequence in the presence of a renalgenerative agent (i.e., spinal cord tissue) as shown in Tables 1 and 2.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations. Suitable control nucleic acids can readily be determined by one of ordinary skill in the art.

In some embodiments, the test cell population is compared to multiple reference cell populations. Each of the multiple reference populations may differ in the known parameter. Thus, a test cell population may be compared to a first reference cell population known to have been exposed to a renalgenerative agent, as well as a second reference population known have not been exposed to a renalgenerative agent.

The test cell population that is exposed to, i.e., contacted with, the test renalgenerative agent can be any number of cells, i. e., one or more cells, and can be provided in vitro, in vivo, or ex vivo.

In other embodiments, the test cell population can be divided into two or more subpopulations. The subpopulations can be created by dividing the first population of cells to create as identical a subpopulation as possible. This will be suitable, in, for example, in vitro or ex vivo screening methods. In some embodiments, various sub populations can be exposed to a control agent, and/or a test agent, multiple test agents, or, e.g., varying dosages of one or multiple test agents administered together, or in various combinations.

Preferably, cells in the reference cell population are derived from a tissue type as similar as possible to test cell, e.g., renal cells (i.e., epithelial, endothelial or mesangial) such nephrons or glomeruli or mesenchyme such as metanephric, pronephric or mesonephric. In some embodiments, the control cell is derived from the same subject as the test cell, e.g., from a region proximal to the region of origin of the test cell. In other embodiments, the reference cell population is derived from a plurality of cells. For example, the reference cell population can be a database of expression patterns from previously tested cells for which one of the herein-described parameters or conditions is known.

The subject is preferably a mammal. The mammal can be, e.g., a human, non-human primate, mouse, rat, dog, cat, horse, or cow.

Screening for Renalregenterative Agents

In one aspect, the invention provides a method screening for renalgenerative agents. By “renalgenerative agent” is meant an agent that promotes renal development (e.g., kidney organogenesis), renal growth and renal repair following injury. The renalgenerative agent can be identified by providing a cell population that includes cells capable of expressing one or more nucleic acid sequences homologous to those listed in Tables 1 and 2 as MET 1-245. The sequences need not be identical to sequences including MET 1-245 so as long as the sequence is sufficiently similar that specific hybridization can be detected. Preferably, the cell includes sequences that are identical, or nearly identical to those identifying the MET nucleic acids shown in Tables 1 and 2.

Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent, parathyroid hormone

An alteration in expression of the nucleic acid sequence in the test cell population compared to the expression of the nucleic acid sequence in the reference cell population that has not been exposed to the test agent indicates the test agent is a renalgenerative agent.

The invention also includes the renalgenerative agent identified according to this screening method, and a pharmaceutical composition which includes the renalgenerative agent.

Assessing Renalgenerative Activity of an Agent in a Subject

The differentially expressed MET sequences identified herein also allow for the renalgenerative activity of a renalgenerative agent to be determined or monitored. In this method, a test cell population from a subject is exposed to a test agent, i.e. a. renalgenerative agent. If desired, test cell populations can be taken from the subject at various time points before, during, or after exposure to the test agent. Expression of one or more of the MET sequences, e.g., MET 1-245, in the cell population is then measured and compared to a reference cell population which includes cells whose exposure status to a renalgenerative agent is known. Preferably, the reference cells not been exposed to the test agent.

If the reference cell population contains no cells exposed to the treatment, a similarity in expression between MET sequences in the test cell population and the reference cell population indicates that the treatment is non-renalgenerative. However, a difference in expression between MET sequences in the test population and this reference cell population indicates the treatment is renalgenerative.

Methods of Treating Renal Disorders or Modulating Kidney Organogenesis in a Subject

Also included in the invention is a method of treating, i.e., preventing or delaying the onset of a renal disorder or modulating kidney organogenesis in a subject by administering to the subject an agent which modulates the expression or activity of one or more nucleic acids or polypeptides selected from the group consisting of MET 1-245 “Modulates” is meant to include increase or decrease expression or activity of the MET polypeptides or nucleic acids. Additionally, the invention provides methods of treating, i.e., preventing or delaying the onset of a renal disorder or modulating kidney organogenesis in a subject by administering to the subject one or more MET polypeptides of nucleic acids Preferably, modulation results in alteration alter the expression or activity of the MET genes or gene products in a subject to a level similar or identical to a subject not suffering from the renal disorder.

The renal disorder can be any of the pathophysiologies described herein, such as, kidney cancer, e.g., renal cell carcinoma, Wilm's tumor, or transitional cell carcinoma, agenesis, nephropathy, e.g, diabetic, glomerular disease, e.g., infection related and glomerlosclerosis, nephritis, e.g., lupus nephritis, and hereditary nephritis, or renal failure, e.g., acute and chronic. The subject can be, e.g., a human, a rodent such as a mouse or rat, or a dog or cat.

The herein described MET nucleic acids, polypeptides, antibodies, agonists, and antagonists when used therapeutically are referred to herein as “Therapeutics”. Methods of administration of Therapeutics include, but are not limited to, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the Therapeutic into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

Various delivery systems are known and can be used to administer a Therapeutic of the present invention including, e.g: (i) encapsulation in liposomes, microparticles, microcapsules; (ii) recombinant cells capable of expressing the Therapeutic; (iii) receptor-mediated endocytosis (See, e.g., Wu and Wu, 1987. J Biol Chem 262:4429-4432); (iv) construction of a Therapeutic nucleic acid as part of a retroviral or other vector, and the like. In one embodiment of the present invention, the Therapeutic may be delivered in a vesicle, in particular a liposome. In a liposome, the protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028; and 4,737,323, all of which are incorporated herein by reference. In yet another embodiment, the Therapeutic can be delivered in a controlled release system including, e.g: a delivery pump (See, e.g., Saudek, et al., 1989. New Engl J Med 321:574 and a semi-permeable polymeric material (See, e.g., Howard, et al., 1989. J Neurosurg 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, Fla.).

In a specific embodiment of the present invention, where the Therapeutic is a nucleic acid encoding a protein, the Therapeutic nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, e.g., Joliot, et al., 1991. Proc Natl Acad Sci USA 88:1864-1868), and the like. Alternatively, a nucleic acid Therapeutic can be introduced intracellularly and incorporated within host cell DNA for expression, by homologous recombination.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the Therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. However, suitable dosage ranges for intravenous administration of the Therapeutics of the present invention are generally about 20-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Polynucleotides of the present invention can also be used for gene therapy. Gene therapy refers to therapy that is performed by the administration of a specific nucleic acid to a subject. Delivery of the Therapeutic nucleic acid into a mammalian subject may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. Polynucleotides of the invention may also be administered by other known methods for introduction of nucleic acid into a cell or organism (including, without limitation, in the form of viral vectors or naked DNA). Any of the methodologies relating to gene therapy available within the art may be used in the practice of the present invention. See e.g., Goldspiel, et al., 1993. Clin Pharm 12:488-505.

Cells may also be cultured ex vivo in the presence of therapeutic agents or proteins of the present invention in order to proliferate or to produce a desired effect on or activity in such cells. Treated cells can then be introduced in vivo for therapeutic purposes.

Methods of Modulating Kidney Organogenesis

Also included in the invention is a method o modulating kidney organogenesis contacting a cell, e.g., kidney cell, or mesenchyme, e.g., metanephric mesenchyme with an agent which modulates the expression or activity of one or more nucleic acids or polypeptides selected from the group consisting of MET 1-245. “Modulates” is meant to include increase or decrease expression or activity of the MET nucleic acids or polypeptides.

The cell population that is exposed to, i.e., contacted with, the agent can be any number of cells, i.e., one or more cells, and can be provided in vitro, in vivo, or ex vivo.

Screening Assays for Identifying a Candidate Therapeutic Agent for Treating or Preventing Renal Disorders

The differentially expressed sequences disclosed herein can also be used to identify candidate therapeutic agents for treating renal disorders. The method is based on screening a candidate therapeutic agent to determine if it induces an expression profile of one or more MET 1-245 sequences, sequences in a test cell population.

In the method, a test cell population is exposed to a test agent or a combination of test agents (sequentially or consequentially), and the expression of one or more of the MET sequences is measured. The expression of the MET sequences in the test population is compared to expression level of the MET sequences in a reference cell population whose renalgenerative agent status is known. If the reference cell population contains cells that have not been exposed to a renalgenerative agent, alteration of expression of the nucleic acids in the test cell population as compared to the reference cell population indicates that the test agent is a candidate therapeutic agent.

In some embodiments, the reference cell population includes cells that have been exposed to a test agent. When this cell population is used, an alteration in expression of the nucleic acid sequences in the presence of the agent from the expression profile of the cell population in the absence of the agent indicates the agent is a candidate therapeutic agent. In other embodiments, the test cell population includes cells that have not been exposed to a renalgenerative agent. For this cell population, a similarity in expression of the MET sequences in the test and control cell populations indicates the test agent is not a candidate therapeutic agent, while a difference suggests it is a candidate.

The test agent can be a compound not previously described or can be a previously known compound but which is not known to renalgenerative

An agent effective in stimulating expression of underexpressed genes, or in suppressing expression of overexpressed genes can be further tested for its ability to prevent the renal disorder and as a potential therapeutic useful for the treatment of such pathophysiology.

Selecting a Therapeutic Agent for Treating a Renal Disorder that is Appropriate for a Particular Individual

Differences in the genetic makeup of individuals can result in differences in their relative abilities to metabolize various drugs. An agent that is metabolized in a subject to act as a renalgenerative agent can manifest itself by inducing a change in gene expression pattern from that characteristic of a pathophysiologic state to a gene expression pattern characteristic of a non-pathophysiologic state. Accordingly, the differentially expressed MET sequences disclosed herein allow for a putative therapeutic or prophylactic agent to be tested in a test cell population from a selected subject in order to determine if the agent is a suitable renalgenerative agent in the subject.

To identify a renalgenerative agent, that is appropriate for a specific subject, a test cell population from the subject is exposed to a therapeutic agent, and the expression of one or more of MET 1-245 sequences is measured.

In some embodiments, the test cell population contains an renal cell,. In other embodiments, the agent is first mixed with a cell extract, e.g., a renal cell extract, which contains enzymes that metabolize drugs into an active form. The activated form of the therapeutic agent can then be mixed with the test cell population and gene expression measured. Preferably, the cell population is contacted ex vivo with the agent or activated form of the agent.

Expression of the nucleic acid sequences in the test cell population is then compared to the expression of the nucleic acid sequences a reference cell population. The reference cell population includes at least one cell whose renalgenerative agent status is known. If the reference cell had been exposed to a renalgenerative agent a similar gene expression profile between the test cell population and the reference cell population indicates the agent is suitable for treating the pathophysiology in the subject. A difference in expression between sequences in the test cell population and those in the reference cell population indicates that the agent is not suitable for treating the renal disorder in the subject.

If the reference cell has not been exposed to a renalgenerative agent, a similarity in gene expression patterns between the test cell population and the reference cell population indicates the agent is not suitable for treating the renal disorder in the subject, while a dissimilar gene expression patterns indicate the agent will be suitable for treating the subject.

In some embodiments, a decrease in expression of one or more of the sequences MET:1-245 or an increase in expression of one or more of the sequences MET:1-245 in a test cell population relative to a reference cell population is indicative that the agent is therapeutic.

The test agent can be any compound or composition. In some embodiments the test agents are compounds and composition know to be renalgenerative agents.

Methods of Diagnosing or Determining the Susceptibility of a Renal Disorder in a Subject

The invention further provides a method of diagnosing a renal disorder. A disorder is diagnosed by examining the expression of one or more MET nucleic acid sequences from a test population of cells from a subject suspected of have the disorder.

Expression of one or more of the MET nucleic acid sequences, e.g. MET:1-245 is measured in the test cell and compared to the expression of the sequences in the reference cell population. The reference cell population contains at least one cell whose, or disease status (i.e., the reference cell population is from a subject suffering from a renal disorder) is known. If the reference cell population contains cells that have not suffering from a renal disorder, then a similarity in expression between MET sequences in the test population and the reference cell population indicates the subject does not have a renal disorder. A difference in expression between MET sequences in the test population and the reference cell population indicates the reference cell population has a renal disorder.

Conversely, when the reference cell population contains cells that have a renal disorder, a similarity in expression pattern between the test cell population and the reference cell population indicates the test cell population has a renal disorder. A difference in expression between MET sequences in the test population and the reference cell population indicates the subject does not have a renal disorder.

Assessing Efficacy of Treatment of a Renal Disorder in a Subject

The differentially expressed MET sequences identified herein also allow for the course of treatment of a renal disorder to be monitored. In this method, a test cell population is provided from a subject undergoing treatment for renal disorder. If desired, test cell populations can be taken from the subject at various time points before, during, or after treatment. Expression of one or more of the MET sequences, e.g., MET: 1-245 in the cell population is then measured and compared to a reference cell population which includes cells whose pathophysiologic state is known. Preferably, the reference cells not been exposed to the treatment.

If the reference cell population contains no cells exposed to the treatment, a similarity in expression between MET sequences in the test cell population and the reference cell population indicates that the treatment is efficacious. However, a difference in expression between MET sequences in the test population and this reference cell population indicates the treatment is not efficacious.

By “efficacious” is meant that the treatment leads to a decrease in the pathophysiology in a subject. When treatment is applied prophylactically, “efficacious” means that the treatment retards or prevents a pathophysiology.

Efficaciousness can be determined in association with any known method for treating the particular pathophysiology.

Kits and Nucleic Acid Collections for Identifying MET Nucleic Acids

In another aspect, the invention provides a kit useful for examining a renal disorders, and renalgenerative agents. The kit can include nucleic acids that detect two or more MET sequences. In preferred embodiments, the kit includes reagents which detect 3, 4, 5, 6, 8, 10, 12, 15, 20, 25, 30, 35, 50, 100, 150, 200 or all of the MET nucleic acid sequences.

The invention also includes an isolated plurality of sequences which can identify one or more MET responsive nucleic acid sequences.

The kit or plurality may include, e.g., sequence homologous to MET nucleic acid sequences, or sequences which can specifically identify one or more MET nucleic acid sequences.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of screening for a renalgenerative agent, the method comprising;
(a) providing a test cell population comprising a cell capable of expressing one or more nucleic acid sequences selected from the group consisting of MET: 1-1-244 and 245;
(b) contacting the test cell population with a test agent;
(c) measuring expression of one or more of the nucleic acid sequences in the test cell population;
(d) comparing the expression of the nucleic acid sequences in the test cell population to the expression of the nucleic acid sequences in a reference cell population comprising at least one cell whose renalgenerative agent exposure status to is known; and
(e) identifying a difference in expression levels of the MET sequence, if present, in the test cell population and reference cell population,
thereby screening for a renalgenerative agent.

2. The method of claim 1, wherein the method comprises comparing the expression of 40 or more of the nucleic acid sequences.

3. The method of claim 1, wherein the expression of the nucleic acid sequences in the test cell population is decreased as compared to the reference cell population.

4. The method of claim 1, wherein the expression of the nucleic acid sequences in the test cell population is increased as compared to the reference cell population.

5. The method of claim 1, wherein the test cell population is provided in vitro.

6. The method of claim 1, wherein the test cell population is provided ex vivo from a mammalian subject.

7. The method of claim 1, wherein the test cell population is provided in vivo in a mammalian subject.

8. The method of claim 1, wherein the test cell population is derived from a human or rodent subject.

9. The method of claim 1, wherein the test cell population includes a renal cell.

10. A method of assessing the renalgenerative activity of a test agent in a subject, the method comprising:
(a) providing from the subject a test cell population comprising a cell capable of expressing one or more nucleic acid sequences selected from the group consisting of MET: 1-244 and 245;
(b) contacting the test cell population with a test agent;
(c) measuring expression of one or more of the nucleic acid sequences in the test cell population; and
(d) comparing the expression of the nucleic acid sequences in the test cell population to the expression of the nucleic acid sequences in a reference cell population comprising at least one cell whose renalgenerative agent exposure status is known;
(e) identifying a difference in expression levels of the nucleic acid sequences, if present, in the test cell population and the reference cell population,
thereby assessing the renalgenerative activity of the test agent in the subject.

11. The method of claim 10, wherein the expression of the nucleic acid sequences in the test cell population is decreased as compared to the reference cell population.

12. The method of claim 10, wherein the expression of the nucleic acid sequences in the test cell population is increased as compared to the reference cell population.

13. The method of claim 10 wherein said subject is a human or rodent.

14. The method of claim 10, wherein the test cell population is provided ex vivo from said subject.

15. The method of claim 10, wherein the test cell population is provided in vivo from said subject.

16. A method of treating a renal disorder in a subject, the method comprising administering to the subject an agent that modulates the expression or the activity of one or more nucleic acids selected from the group consisting of MET: 1-244 and 245.

17. The method of claim 16, wherein the renal disorder is a renal formation disorder.

18. The method of claim 16, wherein the renal disorder is a renal cell carcinoma.

19. The method of claim 16, wherein the renal disorder is a selected from the group comprising nephritis, nephropathy, and glomerular disease.

20. A method of modulating kidney organogenesis, the method comprising contacting a cell with an agent that modulates the expression or the activity of one or more nucleic acids selected from the group consisting of MET: 1-244 and 245.

21. A kit which detects two or more of the nucleic acid sequences selected from the group consisting of MET: 1-245.

22. An array which detects one or more of the nucleic acid selected from the group consisting of MET: 1-245.

23. A plurality of nucleic acid comprising one or more of the nucleic acid selected from the group consisting of MET: 1-245.
